(19) United States
(12) Patent Application Publication
(10) Pub. No.: US 2016/0287770 A1
(43) Pub. Date: Oct. 6, 2016

(54) IMPLANTABLE CONNECTOR ASSEMBLY AND METHOD OF COMMUNICATING AN ELEMENT TO AN IMPLANTABLE DEVICE

(71) Applicant: ODU GmbH & Co. KG, Muehldorf a. Inn (DE)

(72) Inventor: Josef Weigand, Heldenstein (DE)

(21) Appl. No.: 14/676,426

(22) Filed: Apr. 1, 2015

(51) Int. Cl. A61M 1/10; H01R 27/02; H01R 13/52

(52) U.S. Cl. CPC A61M 1/1008; H01R 13/5202; H01R 27/02

(57) ABSTRACT

An implantable connector assembly (14) for communicating an element to an implantable device (12) within a patient (20) comprises a plug (16), a receptacle (18), and a pair of communication structures (72, 108). The plug (16) includes a plunger body (64). The receptacle (18) includes a sleeve (98) and a stopper (114). The sleeve (98) defines an opening (104), and the stopper (114) is resiliency mounted within the sleeve (98) to cover and fluidly seal the opening (104). The pair of communication structures (72, 108) is positioned respectively on the plunger body (64) and the sleeve (98). One of the communication structures (72, 108) connects to a source and the other communication structure (108, 72) connects to the implantable device (12). The plunger body (64) inserts into the sleeve (98) to displace the stopper (114) and couple the pair of communication structures (72, 108) for communication of the element therebetween.

### TECHNICAL FIELD

The present invention relates generally to an apparatus and method for communicating an element from a source to an implantable medical device within the patient.

Implantable medical devices, such as heart pumps, pacemakers, controllers, batteries, catheters, or drug delivery pumps are well known in the medical arts to improve patient outcomes. These implantable medical devices are surgically positioned within the patient's body and may reside in a subcutaneous pocket during the life of the patient or useful life of the device. During this time, it may be necessary to provide an element, such as an electrical power or medical fluid, to the device within the subcutaneous pocket from a supply source exterior of the patient. For this reason, a cable for communicating these elements connects to the device, extends through the subcutaneous pocket, and terminates outside of the patient for accessing the device.

Traditionally, the connection between the cable and the device is permanently and fluidly sealed to inhibit contaminants from entering the device or cable during use. Such contamination may result from bodily fluids or any foreign matter used during surgery and cause reduced performance or failure of the device. While generally effective at inhibiting harmful contamination during use, the cable extending from the implantable device during surgery tends to block access to the patient. Moreover, depending on the position of the subcutaneous pocket, positioning the implantable device within the pocket may be relatively complex since a surgeon must handle both the implantable device and the cable.

There is a need for an apparatus and method that effectively communicates an element from a source to an implantable medical device while addressing issues such as those discussed above.

### SUMMARY

An exemplary embodiment of an apparatus for implantation within a patient comprises an implantable device, a cable, and an implantable connector assembly. The implantable device is configured for being positioned and operating at least subcutaneously within the patient. The cable is configured for communicating an element from a source exterior of a patient or implanted within the patient toward the implantable device within the patient. The implantable connecting assembly includes a plug, a receptacle, and a pair of communication structures. The plug is operatively connected to one of the cable and the implantable device and includes a plunger body. The receptacle is operatively connected to the other of the cable and the implantable device. The receptacle includes a sleeve and a stopper. The sleeve defines an opening sized for receiving the plunger body. The stopper is resiliency mounted within the sleeve and biased toward the opening to cover and fluidly seal the opening for inhibiting contamination from entering within the sleeve when the plunger body is withdrawn from the sleeve. The pair of communication structures is positioned respectively on the plunger body and the sleeve. One of the pair of communication structures operatively connects to the cable for receiving the element from the cable. The other of the pair of communication structures operatively connects to the implantable device for directing the element to the implantable device. Accordingly, the plunger body inserts into the sleeve to displace the stopper and removably couple the pair of communication structures for communication of the element therebetween.

An exemplary embodiment of an implantable connector assembly for communicating an element from a source exterior of a patient or implanted within the patient to an implantable device within the patient comprises a plug, a receptacle, and a pair of communication structures. The plug is configured to operatively connect to one of the source and the implantable device and includes a plunger body. The receptacle is configured to operatively connect to the other of the source and the implantable device and includes a sleeve and a stopper. The sleeve defines an opening steed for receiving the plunger body. The stopper is resiliently mounted within the sleeve and biased toward the opening to cover and fluidly seal the opening for inhibiting contamination from entering the sleeve when tie plunger body is withdrawn from the sleeve. The pair of communication structures is positioned respectively on the plunger body and the sleeve. One of the pair of communication structures is configured to operatively connect to the source for receiving the element from the source, and the other of the pair of communication structures is configured to operatively connect to the implantable device for directing the element to the implantable device. Accordingly, the plunger body inserts into the sleeve to displace the stopper and removably couple the pair of communication structures for communication of the element therebetween.

In use, the implantable connector assembly communicates an element from a source to an implantable device within the patient by inserting a plunger body of a plug Into a sleeve of a receptacle. The method includes displacing a stopper with the plunger body to unseal an opening in the sleeve. Furthermore, the method includes connecting a communication structure of the plunger body with a communication structure of the sleeve through the opening. The method also includes fluidly sealing the plug against the receptacle to inhibit contamination of the communication structures.

Various additional objectives, advantages, and features of the invention will be appreciated from a review of the following detailed description of the illustrative embodiments taken in conjunction with the accompanying drawings.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1 is a diagrammatic view a circulatory assist system having an apparatus with an embodiment of an implantable connector assembly removably coupled within a patient.

FIG. 2A is a cross-sectional view of the implantable connector assembly of the apparatus shown in FIG. 1.

FIG. 2B is similar to FIG. 2A, but shows the implantable connector assembly decoupled.

### DETAILED DESCRIPTION

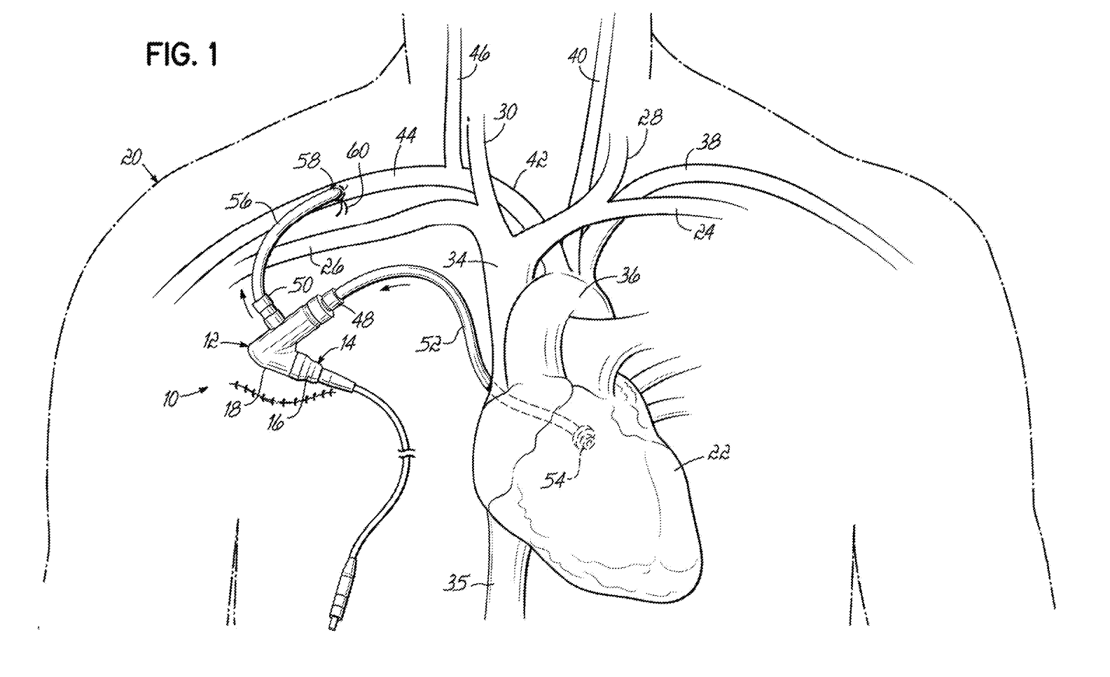

FIG. 1 illustrates an implanted circulatory assist system 10 having an implantable device 12, such as a pump, with an exemplary embodiment of an implantable connector assembly 14. The implantable connector assembly 14 includes a plug 16 cooperating with a receptacle 18 that reduces contamination resulting from removable connection within a patient 20. As used herein, the term “contamination” refers to any bodily fluids or foreign matter that may reduce or destroy communication of an element from the plug 16 to the receptacle 18 if introduced within the implantable connector assembly 14. For illustrative purposes, certain anatomy is shown including a heart 22 of the patient 20 aided by the pump. However, any implantable device 12 may be positioned at least subcutaneously within the patient 20 and include the implantable connector assembly 14 to operatively connect the implantable device 12 to a source, such as a supply source (not shown) exterior of the patient 20. For example, the implantable device 12 may be in the form of the pump shown in FIG. 1 or, alternatively, may be any other implantable medical device including, but not limited to, a pacemaker

and operate generally as understood in the current state of the art. While these alternative implantable devices 12 may be positioned elsewhere within the body and configured for alternative operations, it will be appreciated that the implantable connector assembly 14 may be similarly used for communicating one or more elements from exterior of the patient 20 to any implantable device 12 within the patient 20 or between a plurality of implantable devices 12 within the patient 20. As used herein, the term “element” generally refers to any one of an electrical signal, a packet of information, electrical power, and a fluid, such as a liquid or a gas, that may be communicated to the implantable device 12 via the implantable connector assembly 14. Accordingly, the term “element” is not intended to limit the invention to any particular type of communicant passing through the implantable connector assembly 14.

To power the implantable device 12, a cable 62 is removably connected to the implantable device 12 via the implantable connecter assembly 14 within the patient 20. The cable 62 may extend transdermally from the implantable device 12 to a position in the abdomen where the cable 62 exits the patient 20 and connects to a power supply source (not shown). Suitable power supplies may be any universal-type power supply that sends electrical power to the implantable device 12 through the cable 62 and the implantable connector assembly 14.

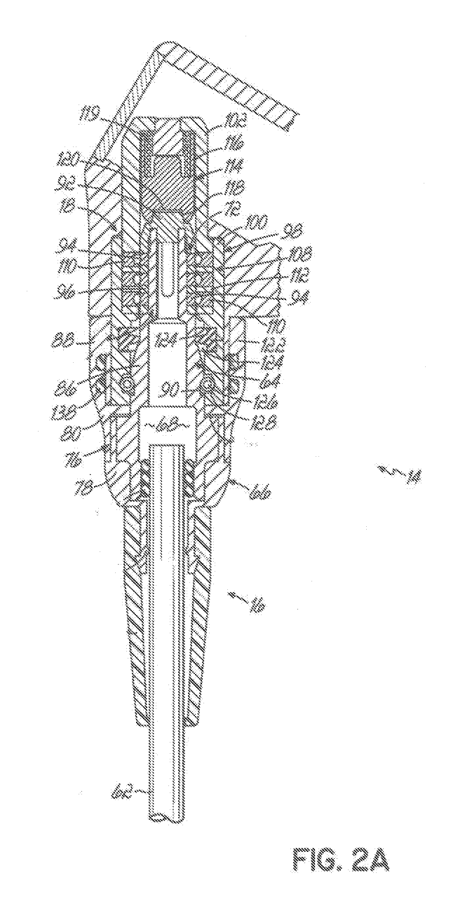

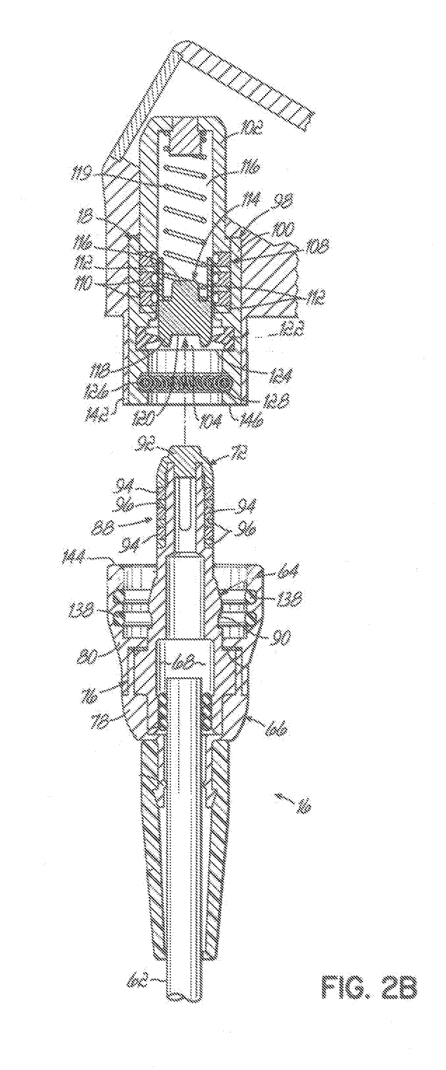

FIG. 2A and FIG. 2B show the implantable connector assembly 14 having the plug 16 and the receptacle 18 in greater detail. The plug 16 connects to the cable 62 defining a generally flexible electrical conduit (not shown) for communicating the electrical power to the plug 16. As such, the exemplary embodiment of the implantable connector assembly 14 removably connects the plug 16 directly into the receptacle 18.

However, it will be appreciated that the plug 16 and the receptacle 18 may be reversed such that the plug 16 is integrated into the implantable device 12 and the receptacle 18 is connected to the cable 62. It will be further appreciated that either the receptacle 18, or alternatively the plug 16, need not be directly integrated into the implantable device 12. For example, the receptacle 18, or alternatively the plug 16, may directly connect to another cable (not shown) that, in turn, connects to the implantable device 12.

The plug 16 generally includes a plunger body 64 projecting from the cable 62 and a plug housing 66 at least partially surrounding the cable 62 and the plunger body 64. The cable 62 inserts into a space 68 defined within the plunger body 64.

The space 68 receives electrical wires (not shown) from within the cable 62 for connection to a plug communication structure 72 described below in greater detail.

The plug housing 66 includes an outer plug body 76, each of which is generally hollow for receiving the cable 62 and the plunger body 64. In addition, the outer plug body 76 has a base component 78 and a component 80. The plunger body 64 is rigidly secured within the base component 78 and projects outward from the cable 62 toward the receptacle 18. The base component 78 secures the component 80 in a position proximate to the plunger body 64 and includes a barbed portion 82 securing the cable cover 74 to the cable 62. The base component 78 may be either rigidly or rotatably attached to the component 80.

The plunger body 64 includes a raised portion 86 extending toward an end portion 88. The raised portion 86 defines an annular groove 90 that generally circumscribes the plunger body 64. More particularly, the annular groove 90 and raised portion 86 are annularly surrounded by the coupling component 80, whereas the end portion 88 projects beyond the coupling component 80 toward the receptacle 18. Also, the end portion 88 has a tapered plunger end 92 and the plug communication structure 72. The plunger end 92 opens the receptacle 18 when inserting the plunger body 64 therein such that the plug communication structure 72 may communicate electrical power to the receptacle 18.

According to the exemplary embodiment shown in FIG. 2A and FIG. 2B, the plug communication structure 72 includes a plurality of annular inner electrical conduits 94 electrically isolated from each other by an inner insulator portion 96 of the plunger body 64. The inner electrical conduits 94 receive electrical power from operatively connected electrical wires (not shown), but described briefly above. By way of example, the plug communication structure 72 includes at least two inner electrical conduits 94 and as many as seven inner electrical conduits 94. Alternatively, the plug communication structure 72 includes at least three inner electrical conduits 94 and as many as five inner electrical conduits 94. The plug communication structure 72 may have an outer surface with more than 50% being the inner insulator portion 96 and, more particularly, between 70% and 90% being the inner insulator portion 96 relative to the plurality of inner electrical conduits 94.

The receptacle 18 is configured to removably receive the plug 16 for communicating the electrical power from the plug 16 to the implantable device 12. The receptacle 18 includes a sleeve 98 generally comprised of a coupling tube 100 and an inner tube 102. The inner tube 102 is inserted into the coupling tube 100 and projects outward from the coupling tube 100 toward the implantable device 12. Accordingly, the coupling tube 100 defines an opening 104, and the inner tube 102 defines a bore 106. The inner tube 102 is sized for receiving the end portion 88 of the plunger body 64, while the coupling tube 100 is sized for receiving the raised portion 86 of the plunger body 64. Thus, the opening 104 connects to the pore 106 for receiving the plunger body 64 of the plug 16 within the sleeve 98 of the receptacle 18.

The inner tube 102 also includes a receptacle communication structure 108 configured to cooperate with the plug communication structure 72 for communicating the element, such as the electrical power, therebetween. According to an exemplary embodiment, the receptacle communication structure 108 includes a plurality of annular outer electrical conduits 110 electrically isolated from each other by an outer insulator portion 112 of the inner tube 102. The outer electrical conduits 110 are sized to receive and contact the inner electrical conduits 94 therein for communicating electrical power. The outer electrical conduits 110 are connected to electrical wires (not shown) within the implantable device 12 for delivering electrical power to the remainder of the implantable device 12, such as the pump described above. By way of example, the receptacle communication structure 108 includes at least two outer electrical conduits 110 and as many as seven outer electrical conduits 110. Alternatively, the receptacle communication structure 108 includes at least three outer electrical conduits 110 and as many as five outer electrical conduits 110.

Furthermore, the receptacle 18 includes a resiliency mounted stopper 114 for fluidly sealing the opening 104 and, in turn, inhibiting contamination from entering the bore 106 when the plug 16 is withdrawn from the receptacle 18. The stopper 114 has a recessed end 116 opposite from a sealing end 118. A spring 119 is mounted within the bore 106 and rests within the recessed end 116. Accordingly, the stopper 114 is biased toward the opening 104 to seal the opening 104 with the sealing end 118 when the plug 16 is withdrawn. The sealing end 118 also includes a detent 120 sized for receiving the plunger end 92 when me plunger body 64 is inserted into the sleeve 98 for improved longitudinal and coaxial alignment between stopper 114, the bore 106, and the plunger body 64.

To further fluidly seal the opening 104 and inhibit contamination, the receptacle 18 also includes an annular wiper seal 122 positioned proximate to the opening 104. More particularly, the coupling tube 100 includes an inner seal groove 124 that receives the wiper seal 122 for affixing the wiper seal 122 within the opening 104. The stopper 114, being biased toward the opening 104, contacts the wiper seal 122 with sufficient force to fluidly seal the sealing end 118 of the stopper 114 against the wiper seal 122. As will be described below in greater detail, the wiper seal 122 is also sized to fluidly seal against the end portion 88 of the plunger body 64. Accordingly, the wiper seal 122 further inhibits contamination from entering the opening 104 and bore 106 even when the stopper 114 is displaced longitudinally within the bore 106 by the plunger body 64.

In addition, the coupling tube 100 includes a holding member 126 and an inner holding groove 128. The inner holding groove 128 is annular and extends within the coupling tube 100. The holding member 126 is positioned within the inner holding groove 128 and is sized to engage the annular groove 90 of the plunger body 64 when the plunger body 64 is inserted into the sleeve 98. As shown in the exemplary embodiment of FIG. 2A and FIG. 2B, the holding member 126 is in the form of a toroidal spring that releasably engages the annular groove 90 with enough biasing force to overcome the force of the spring 119 within the bore 106. Thus, the holding member 126 engages the annular groove 90 with sufficient force to hold the plug 16 in a releasably fixed position relative to the receptacle 18. However, it will be appreciated that the holding member 126 may be any annular component sized for the inner holding groove 128 that provides sufficient biasing force for engaging the annular groove 90.

In use, the receptacle 18 and implantable device 12 are implanted at least subcutaneously within the patient 20 (see FIG. 1) without the plug 16 and the remainder of the cable 62 connected to the receptacle 18. The stopper 114 fluidly seals against the wiper seal 122 to inhibit contamination from entering the opening 104 and contaminating the receptacle communication structure 108 within the bore 106. Once implanted, the plunger body 64 of the plug 16 is inserted into the sleeve 98 until the plug communication structure 72 and the receptacle communication structure 108 align, contact, and operatively connect for communication of the element therebetween.

As the plunger body 64 is inserted into the sleeve 98, the plunger end 92 coaxially aligns with the detent 120 and the bore 106. The plunger end 92 displaces the stopper 114 longitudinally along the bore 106 and compresses the spring 119 such that the stopper 114 is no longer fluidly sealed against the wiper seal 122. However, as the wiper seal 122 fails to fluidly seal against the stopper 114, the wiper seal 122 fluidly seals against the end portion 88 of the plunger body 64 passing through the opening 104. In this respect, a fluid seal is generally maintained between the patient 20 end receptacle communication structure 108 during the insertion of the plug 16. The wiper seal 122 also tends to reduce contamination on the plunger body 64 and, in particular, the plug communication structure 72, by wiping contamination from the plunger body 64 during insertion of the plug 16.

Once the plug and receptacle communication structures 72, 108 are properly aligned relative to each other, the holding member 126 engages the annular groove 90 in the plug 16. The engagement of the holding member 126 overcomes the biasing spring 119 to removably hold the plug 16 in position relative to the receptacle 18.

In addition, the engagement of the component 80 further fluidly seals the implantable connector assembly 14 from contamination longitudinally with the seals 138. Finally, the element, such as the electrical power, may be provided to the cable 62 and communicated to the implantable device 12 via the implantable connector assembly 14.

The plug 16 is withdrawn from the receptacle 18, and, as such, the stopper 114 again biases against the wiper seal 122 to cover the opening 104 info the bore 106 and fluidly seal the receptacle communication structure 108 from contamination. During the implantation or maintenance of the implantable device 12, it will be appreciated that the insertion and withdrawal of the plug 16 may be repeated as necessary to provide improved access to the patient 20 and reduced surgical complexity (see FIG. 1) by removing the cable 62 from connection with the implantable device 12.

While the present invention has been illustrated by the description of one or more embodiments thereof, and while the embodiments have been described in considerable detail, they are not intended to restrict or in any way limit the scope of the appended claims to such detail. The various features shown and described herein may be used alone or in any combination. Additional advantages and modifications will readily appear to those skilled in the art. The invention in its broader aspects is therefore not limited to the specific details, representative apparatus and method and illustrative examples shown and described. Accordingly, departures may be from such details without departing from the scope of the general inventive concept.

What is claimed is:

1. An apparatus for implantation within a patient, comprising:
an implantable device configured tor being positioned and operating at least subcutaneously within the patient;
a cable configured for communicating an element from a source toward the implantable device within the patient; and
an implantable connector assembly for communicating the element from the cable to the implantable device, comprising:
a plug operatively connected to one of the cable and the implantable device, the plug including a plunger body;
a receptacle operatively connected to the other of the cable and the implantable device, the receptacle including a sleeve and a stopper, the sleeve defining an opening sized for receiving the plunger body, the stopper resiliently mounted within the sleeve and biased toward the opening to cover and fluidly seal the opening for inhibiting contamination from entering within the sleeve when the plunger body is withdrawn from the sleeve; and
a pair of communication structures positioned respectively on the plunger body and the sleeve, one of the pair of communication structures operatively connecting to the cable for receiving the element from the cable and the other of the pair of communication structures operatively connecting to the implantable device for directing the element to the implantable device,
wherein inserting the plunger body into the sleeve displaces the stopper and removably couples the pair of communication structures for communication of the element therebetween.

2. The apparatus of claim 1, wherein the plug is operatively connected to the cable and the receptacle is operatively connected to the implantable device.

3. The apparatus of claim 1 further comprising:
a wiper seal positioned proximate to the opening, the stopper fluidly sealing against the wiper seal to further inhibit the entry of contamination within the sleeve.

4. The apparatus of claim 3, wherein the wiper seal fluidly seals against the plunger body during insertion of the plunger body into the sleeve such that the wiper seal removes further contamination from a portion of the plunger body within the sleeve.

5. The apparatus of claim 1, wherein the plunger body is inserted coaxially within the sleeve.

6. The apparatus of claim 1, wherein the element is an electrical power, and the pair of communication structures are a pair of annular electrical conduits positioned respectively about the plunger body and the sleeve, the pair of annular electrical conduits configured to removably connect for communicating the electrical power therebetween.

7. The apparatus of claim 1, wherein the plug includes a groove, and the receptacle includes a holding member, the groove configured to removably receive the holding member such that the holding member mechanically couples the plug to the receptacle.

8. The apparatus of claim 1 wherein the source is a supply source positioned exterior of the patient.

9. An implantable connector assembly for communicating an element from a source exterior of a patient or implanted within the patient to an implantable device within the patient, comprising:
a plug configured to operatively connect to one of the source and the implantable device, the plug including a plunger body;
a receptacle configured to operatively connect to the other of the source and the implantable device, the receptacle including a sleeve and a stopper, the sleeve defining an opening sized for receiving the plunger body, the stopper resiliently mounted within the sleeve and biased toward the opening to cover and fluidly seal the opening for inhibiting contamination from entering within the sleeve when the plunger body is withdrawn from the sleeve; and
a pair of communication structures positioned respectively on the plunger body and the sleeve, one of the pair of communication structures is configured to operatively connect to the source for receiving the element from the source and the other of the pair of communication structures is configured to operatively connect to the implantable device for directing the element to the implantable device,
wherein inserting the plunger body into the sleeve displaces the stopper and removably couples the pair of communication structures for communication of the element therebetween.

10. The implantable connector assembly of claim 9 further comprising:
a wiper seal positioned proximate to the opening, the stopper fluidly sealing against the wiper seal to further inhibit the entry of contamination within the sleeve.

11. The implantable connector assembly of claim 10, wherein the wiper seal fluidly seals against the plunger body during insertion of the plunger body into the sleeve such that the wiper seal removes further contamination from a portion of the plunger body within the sleeve.

12. The implantable connector assembly of claim 9, wherein the plunger body is inserted coaxially within the sleeve.

13. The implantable connector assembly of claim 9, wherein the element is an electrical power, and the pair of communication structures are a pair of annular electrical conduits positioned respectively about the plunger body and the sleeve, the pair of annular electrical conduits configured to removably connect for communicating the electrical power therebetween.

14. The implantable connector assembly of claim 9, wherein the plug includes a groove, and the receptacle includes a holding member, the groove configured to removably receive the holding member such that the holding member mechanically couples the plug to the receptacle.

15. A method of communicating an element from a source to an implantable device within the patient, comprising:
inserting a plunger body of a plug into a sleeve of a receptacle;
displacing a stopper with the plunger body to unseal an opening within the sleeve;
connecting a communication structure of the plunger body with a communication structure of the sleeve through the opening; and
fluidly sealing the plug against the receptacle to inhibit contamination of the communication structures.

16. The method of claim 15 further comprising:
removing the plunger body of the plug from the sleeve of the receptacle; and
biasing the stopper toward the opening to fluidly sealing the opening to further inhibit entry of the contamination into the sleeve.

17. The method of claim 15 wherein the communication structure of the plunger body and the sleeve is an electrical conduit and the method further comprises:
communicating an electrical power from the electrical conduit of the plunger body to the electrical conduit of the sleeve.

18. The method of claim 15 further comprising:
wiping at least a portion of the plunger body with a wiper seal to remove contamination from the portion of the plunger body.

19. The method of claim 15 further comprising:
aligning the plunger body and the sleeve coaxially.

20. The method of claim 15 wherein the source is a supply source exterior of the patient, and the method further comprises:
communicating the element from the supply source exterior of the patient through the communication structures and to the implanted device within the patient.
